(19) United States
(12) Patent Application Publication
(10) Pub. No.: US 2006/0224414 A1
(43) Pub. Date: Oct. 5, 2006

(54) System and method for providing prescription refills

(76) Inventors: Christopher Berg Astrup, Austin, MN (US); Jeremy Loren Johnson, Austin, MN (US)

Correspondence Address: MERCHANT & GOULD PC, P.O. BOX 2903, MINNEAPOLIS, MN, 55402-0903, US

(21) Appl. No.: 11/096,224

(22) Filed: Mar. 31, 2005

(51) Int. Cl. G06Q 10/00

(52) U.S. Cl. 705/2

(57) ABSTRACT

The invention relates to a system and method for automatically providing prescription refills. In an embodiment, the invention includes a method for filling refill prescriptions for one or more home pharmacies comprising: enrolling the refill prescription records of one or more home pharmacies; calculating an appropriate time to fill refill prescriptions; and filling the refill prescriptions. In an embodiment, the invention includes a system for processing refill prescriptions comprising a hub pharmacy having a hub formulary and a processing unit, and one or more home pharmacies each having a prescription information database comprising records of prescriptions, the home pharmacies in electronic communication with the hub pharmacy.

### FIELD OF THE INVENTION

The invention relates to a system and method for providing pharmacy prescription refills.

### BACKGROUND OF THE INVENTION

Many patients receive prescriptions from health service providers for medications that must be taken over a period of time lasting for months or years. When the prescriptions are filled, such as by a pharmacy, an amount of the medication is typically given to last for only a limited period of time. Therefore, the patient must have their prescription refilled one or more times, typically at regular intervals.

Some pharmacies operate independently while others are a part of large chains. Still other pharmacies can be referred to as mail-order pharmacies because the patient never visits them in-person but rather simply has the filled prescription mailed to them. Large chain pharmacies and mail-order pharmacies generally have competitive advantages over independent pharmacies in at least two ways. First, the large chain pharmacies and mail-order pharmacies are typically more efficient because of their ability to invest in automation technology. Second, because of the large number of prescriptions that they process, large chain pharmacies and mail-order pharmacies can negotiate better third-party contracts and therefore have a lower cost of goods and higher reimbursement rates than smaller independent pharmacies.

Therefore, a need exists for systems and methods that can provide efficiency for independent pharmacies.

### BRIEF DESCRIPTION OF THE DRAWINGS

FIG. 1 is a schematic overview of the interaction between a patient, a home pharmacy, a hub pharmacy, and third parties in accordance with an embodiment of the invention.

FIG. 2 is a schematic overview of the interaction between various third parties.

FIG. 3 is a flowchart showing major tasks that can be performed by, or in association with, embodiments of the invention.

FIG. 4 is a flowchart showing an exemplary process for automatically enrolling prescriptions for processing by a hub pharmacy.

FIG. 5 is a flowchart showing an exemplary process for automatically determining the appropriate time for refilling a prescription

FIG. 6 is a graph showing a hypothetical example of the amount of medication possessed by a patient when prescription refills are provided in accordance with an embodiment of the invention.

FIG. 7 is a graph showing another hypothetical example of the amount of medication possessed by a patient when prescription refills are provided in accordance with an embodiment of the invention.

FIG. 8 is a flowchart showing an exemplary process for filling prescriptions and billing for them.

### SUMMARY OF THE INVENTION

The invention relates to a system and method for providing prescription refills. In an embodiment, the invention includes a method for filling refill prescriptions for one or more home pharmacies comprising: enrolling the refill prescription records of one or more home pharmacies; calculating an appropriate time to fill refill prescriptions; and filling the refill prescriptions. In an embodiment, the invention includes a system for processing refill prescriptions comprising a hub pharmacy having a hub formulary and a processing unit, and one or more home pharmacies each having a prescription information database comprising records of prescriptions, the home pharmacies in electronic communication with the hub pharmacy. In an embodiment of the system, the processing unit is adapted and configured to query the prescription information databases to find records of prescriptions to be refilled. In an embodiment of the system, the hub pharmacy is adapted and configured to fill prescriptions for medications that are on the hub formulary. In an embodiment of the system, the one or more member pharmacies are adapted and configured to fill prescriptions for medications that are not on the hub formulary.

### DETAILED DESCRIPTION

Large chain pharmacies and mail-order pharmacies generally have a competitive advantage over smaller independent pharmacies in at least two ways. First, the large chain pharmacies and mail-order pharmacies are typically more efficient because of their heavy investment in automation technology. These larger pharmacies have the resources to invest in prescription filling systems or robots that allow them to fill prescriptions quickly, accurately, and with less human help. Second, because of the large number of prescriptions that they process, they can negotiate better third-party contracts and get a lower cost of goods and higher reimbursement rates. Further, because they process a much larger number of prescriptions, the amount of inventory they must carry on hand compared with the amount of inventory that they turn over in a given period of time is less than for a smaller independent pharmacy. Thus, large chain pharmacies and mail order pharmacies save money on inventory costs because they have a proportionately smaller amount of inventory on hand relative to the amount they sell over a given period of time.

The present invention provides systems and methods that can allow smaller pharmacies, such as independent pharmacies, to have efficiencies and cost advantages with respect to filling prescriptions. By way of example, in accordance with an embodiment of the invention, one hub pharmacy can serve many home pharmacies. Therefore, in many circumstances, the hub pharmacy purchases a greater amount of inventory than any given home pharmacy normally would and has greater negotiating leverage with suppliers leading to cost advantages. The hub pharmacy may also be able to negotiate better reimbursement rates because of higher volume. Further, the hub pharmacy will have more resources to invest in automation technology so that prescriptions can be filled more efficiently. Therefore, the use of systems and methods according to the invention can allow small independent pharmacies to more effectively compete with larger chain or mail-order pharmacies.

In an embodiment, the invention includes a method for filling refill prescriptions for one or more home pharmacies comprising: enrolling the refill prescription records of one or more home pharmacies; calculating an appropriate time to fill refill prescriptions; and filling the refill prescriptions. In an embodiment, the invention includes a system for processing refill prescriptions comprising a hub pharmacy having a hub formulary and a processing unit, and one or more home pharmacies each having a prescription information database comprising records of prescriptions, the home pharmacies in electronic communication with the hub pharmacy.

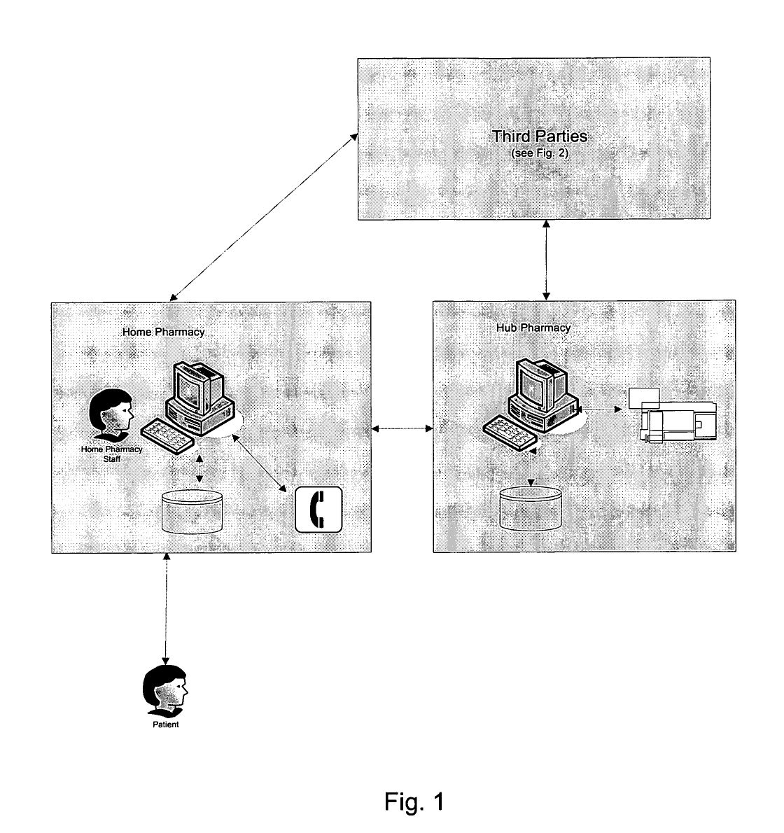

Referring now to FIG. 1, a schematic overview of an exemplary interaction between a patient, a home pharmacy, a hub pharmacy, and a third party payer in accordance with an embodiment of the invention is shown. The patient interacts with the home pharmacy and provides a prescription to be filled. A prescription may be dropped off, called in, or faxed by either the patient or the health care provider. The home pharmacy fills the original prescription and inputs information about the prescription into their computer system or other record keeping device. The prescription records of the home pharmacy are queried to find prescriptions having refills that need to be filled. The query may be run weekly or at any given desired interval of time. Using logic described in greater detail below, the hub pharmacy either fills the prescription or causes the home pharmacy to fill the prescription and also provides billing information to a third party. The hub pharmacy delivers prescriptions to the home pharmacy and optionally notifies the patient that the prescription is ready to be picked-up.

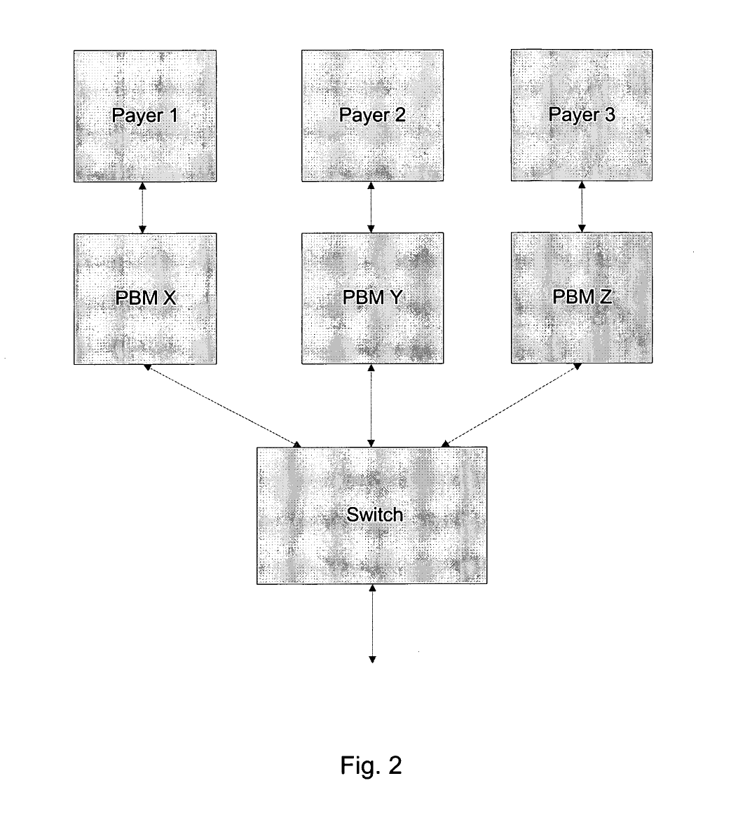

FIG. 2 shows the relationship between various third parties that are involved in processing prescription benefit claims. Generally, third party payers, such as insurance companies or self-insured corporations, work with pharmacy benefit managers (PBMs) to administer claims for prescription drugs. PBMs may require information to be submitted in a manner and format that is specific to them and different than that required by other PBMs. A “Switch” is a company that acts as an intermediary between a pharmacy and the PBMs. Insurance claims are sent electronically to the Switch, and the Switch then electronically sends the claims to the correct PBM. Information on if the claim was paid or rejected is then sent back to the Switch, and then from the Switch to the pharmacy. An exemplary Switch is eRX, Fort Worth, Tex. However, a Switch can also be integrated into the system of the hub pharmacy.

As used herein, the term “home pharmacy” refers to a pharmacy, such as an independent pharmacy, that uses prescription filling services of the systems and/or methods of the invention to fill prescriptions for patients. Home pharmacies can include pharmacies of all types including both independent pharmacies and chain pharmacies. In many embodiments, home pharmacies are at distinct geographical locations different from one another and different from the hub pharmacy.

As used herein, the term “hub pharmacy” refers to a pharmacy that interacts with, or forms part of, the system of the invention in order to provide prescription-filling services for home pharmacies. Components of a “hub pharmacy” may all be physically located in one geographic place or may be located in a plurality of locations.

As used herein, the term “hub pharmacy formulary” refers to a list of all the medications that are in the inventory of the hub pharmacy for filling prescriptions.

System and Method Overview

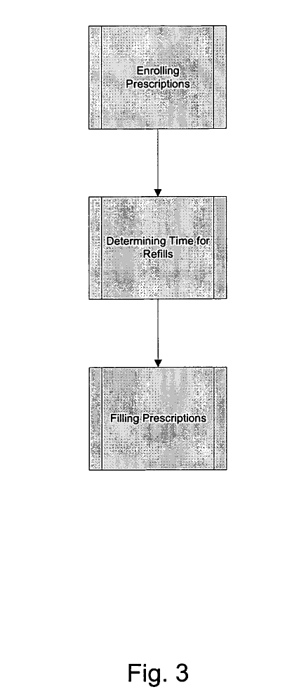

Referring now to FIG. 3, a flowchart is provided showing major tasks that can be performed by, or in association with, the system and methods of the invention. Embodiments of the system can perform various tasks including: 1.) Enrolling prescriptions from the databases or information kept by independent pharmacies, 2.) Determining the appropriate time for prescriptions to be refilled, and 3.) Filling prescriptions. Examples of how these tasks can be performed will now be described in greater detail. However, one of skill in the art will appreciate that other specific ways of performing these tasks may also be used.

Prescription Enrollment

Prescriptions for medications can be enrolled for processing by the systems and methods of the invention in various ways. In an embodiment, prescriptions can be enrolled manually by pharmacists or other workers at home pharmacies (pharmacy staff). In an embodiment, the systems and/or methods of the invention can enroll prescriptions from home pharmacies automatically.

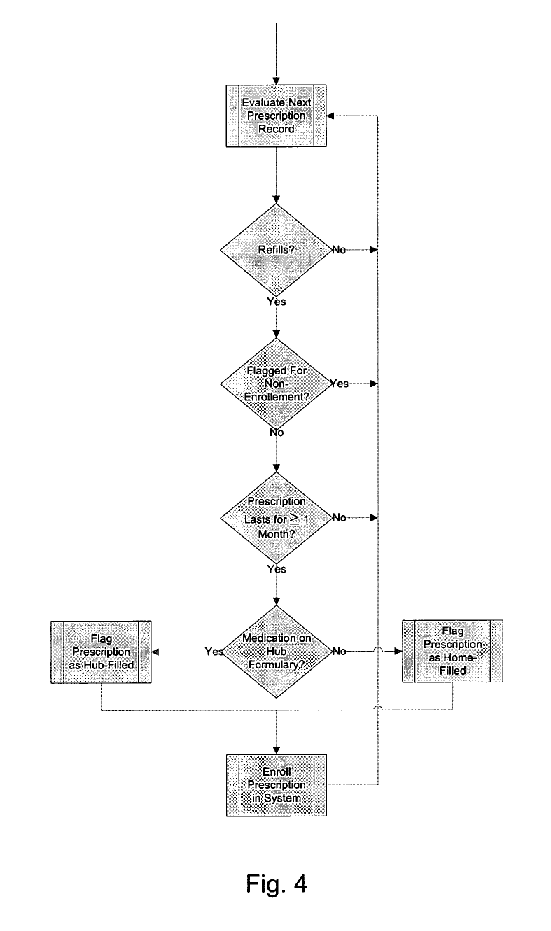

Referring now to FIG. 4, a flowchart of a process for automatically enrolling prescriptions from pharmacies is shown. The system of the invention can query the database or records kept by the home pharmacy. The system of the invention does not depend on the home pharmacy operating a particular software package or computer system. Rather, the system of the invention can work with a home pharmacy regardless of the type of pharmacy software and/or hardware packages it has. By way of example, the system of the invention can work with home pharmacies having systems including CarePoint, Viking, QS/1, CRx, PDX, HCC, Jascorp, SXC, KeyCentrix, Lake Country Systems, Rx30, Etreby, and the like.

In an exemplary process, the system starts to evaluate the prescription records serially. In an embodiment, all records of prescriptions are searched. In an embodiment, only records that have been recently added to the database are searched. The system performs a first query, such as determining if there are any remaining refills for the prescription. If there are no remaining refills, the system then moves on to evaluate the next prescription record.

In there are remaining refills, the system then performs a second query such as determining whether the specific prescription record being evaluated has been flagged to prevent automated enrollment into the system. If the prescription record has been flagged to prevent automated enrollment into the system, then the system moves on to evaluate the next prescription record, starting from the first query.

If the prescription record has not been flagged, then the system performs a third query such as determining if the prescription is for a days supply≧one month. If the prescription is not for a days supply≧one month, then the system moves on to evaluate the next prescription record, starting from the first query. This query could also be performed for another specific amount of time, for example 28 days.

If the prescription is for a days supply≧one month, then the system performs a fourth query such as comparing the medication that the prescription is written for against the formulary of the hub pharmacy. If the medication is not on the formulary of the hub pharmacy, then this prescription is flagged as such. If the medication is on the formulary of the pharmacy hub, then this prescription is flagged accordingly. In either regard, the prescription record is then enrolled for further processing by the system of the invention.

A greater or lesser number of queries may be performed by embodiments of the invention when enrolling prescriptions from a home pharmacy. Moreover, one of skill in the art will appreciate that the queries may be performed in a different order than described above. Also, prescription records from home pharmacies can be manually enrolled for further processing by the system of the invention such that prescription records can be enrolled even though they do not comply with the queries that may be performed automatically.

The exemplary enrollment process described herein can be performed on a periodic or non-periodic basis. By way of example, the enrollment process can be performed daily, weekly, monthly, or any frequency desired. The enrollment process can also be performed on a non-periodic basis such as on-command as initiated by a user at either the home pharmacy or the hub pharmacy.

In an embodiment, once the decision has been made to enroll the prescription record, data about the prescription can be provided to the hub pharmacy. For example, the data can be transmitted electronically and then stored in a database controlled by the hub pharmacy. However, in an embodiment, all prescription information about a given patient remains in the records of the home pharmacy.

The exemplary enrollment process described herein can be performed as initiated by the home pharmacy or as initiated by the hub pharmacy. In an embodiment, the enrollment process is initiated by the hub pharmacy. Communication, such as electronic communication, between the hub pharmacy and one or more home pharmacies can be conducted through a variety of means well known in the art. By way of example, the hub pharmacy and the home pharmacies can be in electronic communication by way of a virtual private network (VPN), a telephone line, a local area network (LAN), a wide-area network (WAN), a satellite connection, the Internet, or any other wired or wireless means of exchanging data.

In an embodiment, software code to perform the exemplary enrollment process described herein resides on a computer controlled by the home pharmacy. This computer may or may not be physically located at the home pharmacy. In an embodiment, software code to perform this exemplary enrollment process resides on a computer controlled by the hub pharmacy. This computer may or may not be physically located at the hub pharmacy. In an embodiment, software code to perform this exemplary enrollment process resides at a location other than the home pharmacy or the hub pharmacy, such as at a third-party processing center.

In an embodiment, the results of the exemplary enrollment process are put into an extract file. The extract file may then be conveyed to the hub pharmacy in various ways. In an embodiment, the extract file includes information about the prescriptions contained therein such as: store number, prescription number, patient name, patient date of birth, drug directions, drug name, dispensing quantity, patient phone number, last fill date, days supply given on last fill, call back phone number, e-mail address, and the like.

Staff from the home pharmacy can interact with the hub pharmacy in various ways to enter a prescription into the system of the invention or to review or alter information about a prescription already in the system. In an embodiment, the home pharmacy staff uses a web interface to interact with the hub pharmacy. For example, the home pharmacy staff can use a web interface to manually enroll or un-enroll prescriptions from the system. The home pharmacy staff can also use a web interface to review or change details about a prescription enrolled in the system such as changing the dates that prescriptions are scheduled to be filled, the contact method, etc. In an embodiment, the home pharmacy staff enters the home pharmacy number and the prescription number and the website brings up one or more of the following pieces of information about the prescription: Patient Name; Date of Birth; Phone Number; Rx#; Drug Name; NDC#; Drug Directions; Doctor Name; Prescription Expiration Date; Drug Schedule; Drug Manufacturer; Last Fill Date; Last Fill Quantity; Quantity Remaining; Order History; Contact Method (Phone, e-mail, none); Delivery Method (Deliver, Mail, Pickup); and Next Order Date. In an embodiment, the home pharmacy staff can change any of these pieces of information through the web interface.

Determining Prescription Re-Fill Time

Prescriptions for medications can be refilled at various times. In an embodiment, the appropriate time for refilling a prescription can be determined manually. In an embodiment, the appropriate time for refilling a prescription can be determined by the system of the invention. In an embodiment, the appropriate time for refilling a prescription is determined by the system of the invention but is then reviewed and/or modified by a user either at the home pharmacy or the hub pharmacy.

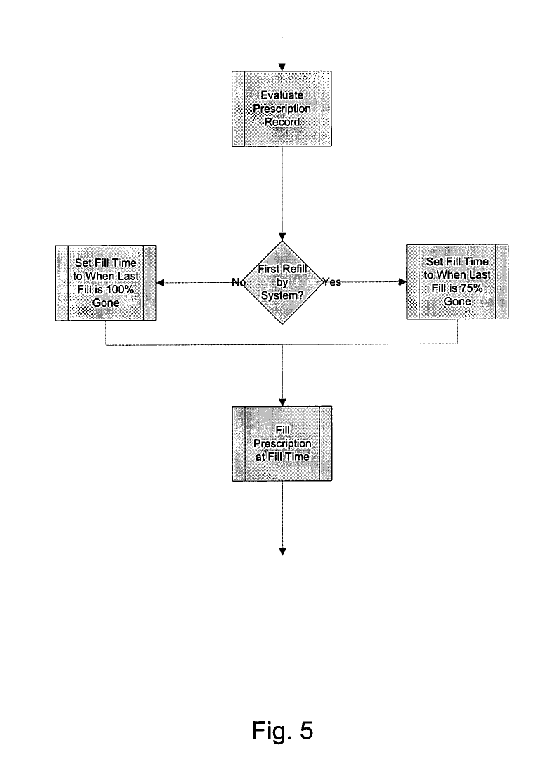

Referring now to FIG. 5, a flowchart of an exemplary process for automatically determining the appropriate time for refilling a prescription is shown. In an embodiment, the system of the invention determines whether or not any refills for the prescription have already been processed by the system of the invention in the past. If the refill is the first re-fill of the prescription to be filled by the system, then the appropriate time for refilling the prescription (fill time) is determined to be when the days supply for the previous fill is about 75% gone. By way of example, if the prescription was for a days supply of 28 days, then the fill time is determined to be when only 7 days supply remain. In other embodiments, the fill time is determined to be when the days supply for the previous fill is about 60%, 65%, 70%, 80%, 85%, 90%, or 95% gone. The precise number may vary based on factors including the nature of the prescription, the rules of the insurer, preferences of the home pharmacy, etc.

If there have been other refills processed by the hub pharmacy, then the fill time is determined to be when the days supply for the previous refill is 100% gone. Therefore, if the previous refill was for a days supply of 28 days, then the fill time is when the 28 days supply is gone. On the assumption that the previous refill was conducted when there was a 7 day supply remaining, then there is also a 7 day supply remaining when the first refill is entirely used up. Therefore, conducting successive refills when the days supply of the previous refills are used up allows the patient to have a reserve amount on hand but does not result in the patient steadily increasing the amount on hand at the end of each successive refill.

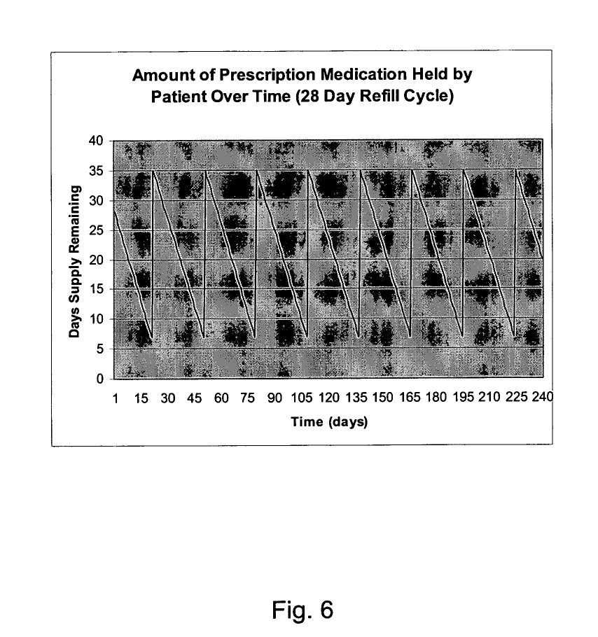

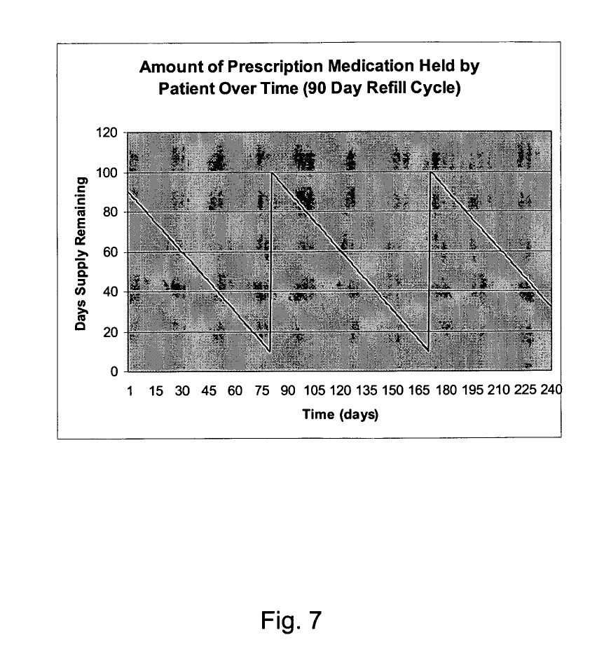

This scheme can be readily understood with reference to FIGS. 6 and 7, which shows hypothetical examples of the amount of medication in the possession of a patient when prescription refills are provided in accordance with an embodiment of the invention. In FIG. 6, the example is for a prescription having a 28 day refill cycle. The patient starts with a 28 day supply on hand and then the first refill is filled after 21 days. When the first refill is provided the supply on hand jumps up to a 35 day supply. However, the subsequent refills are then filled after each additional 28 day time period. Accordingly, supply on hand fluctuates between 7 days and 35 days but does not progressively become higher with each additional refill.

In an embodiment of the invention, the refill is provided a maximum of 10 days prior to the time when the patients supply on hand is exhausted. In FIG. 7, the example is for a prescription having a 90 day refill cycle. After about 67-68 days, the days supply of the prescription is 75% gone, leaving the patient with about a 22-23 day supply remaining. However, since this is more than a 10 day supply, the fill time is determined to be about 80 days after the last fill. Thus, the patient starts with a 90 day supply on hand. After 80 days, the first refill is filled and the supply on hand jumps up to a 100 day supply. However, the subsequent refills are then filled after each additional 90 day time period. Accordingly, supply on hand fluctuates between 10 days and 100 days but does not progressively become higher with each additional refill.

Filling Prescriptions

Embodiments of the invention can fill refill prescriptions automatically. Embodiments of the invention can also process information necessary for billing third parties for prescriptions that have been filled.

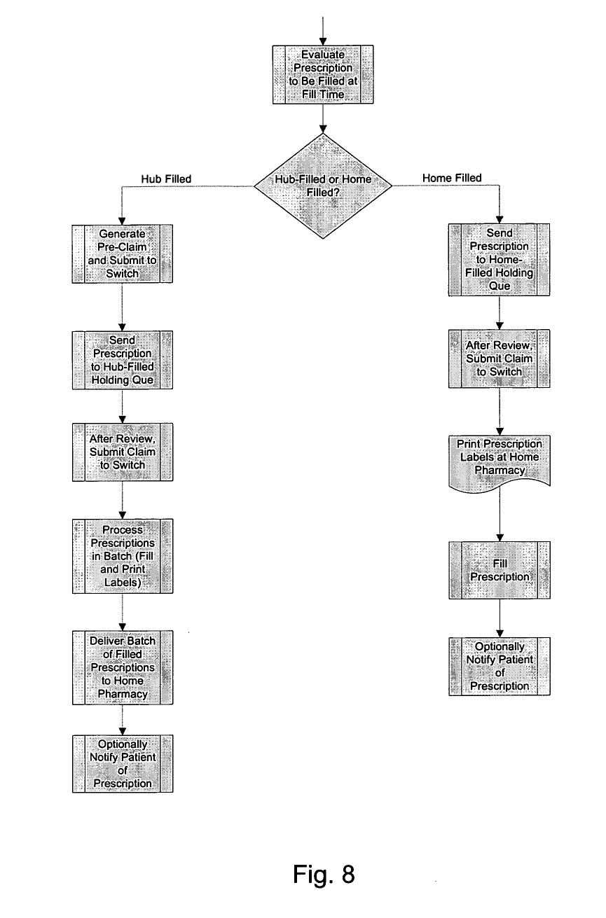

Referring now to FIG. 8, a flowchart of an exemplary process for filling and billing of prescriptions is shown. Each prescription that is enrolled in the system and for which the appropriate time to fill the re-fill prescription has come is evaluated to determine if it will be hub-filled (prepared by the hub pharmacy) or if it will be home-filled (prepared by the home pharmacy). Referring back to FIG. 2, if the prescription is for a medication that was on the hub formulary, then the prescription is a hub-filled prescription. However, if the prescription is for a medication that was not on the hub formulary, then the prescription is a home-filled prescription.

If the prescription is a home-filled prescription, then the information for the re-fill of the prescription is sent to the home pharmacy and held in a home-filled holding queue for further processing by systems or staff of the home pharmacy. This information can include one or more of: Patient Name; Date of Birth; Phone Number; Rx#; Drug Name; NDC#; Drug Directions; Doctor Name; Prescription Expiration Date; Drug Schedule; Drug Manufacturer; Last Fill Date; Last Fill Quantity; and Quantity Remaining. By way of example, a staff person of the home pharmacy may process the prescriptions held in the home-filled holding queue on a periodic basis. Such processing can include performing a review for drug interactions with other prescriptions filled for the specific patient.

After the home-filled prescription is processed by the home pharmacy, billing information for the prescription (insurance claim) is sent to a Switch that in turn submits billing information to the appropriate third party (PBM) using the NCPDP (National Council for Prescription Drug Programs) provider identification number of the home pharmacy. The billing information can be sent to the switch by the home pharmacy directly or by the hub pharmacy.

The switch then conveys information on whether the claim is allowed or denied back to the home pharmacy, either directly or through the hub pharmacy. Then, a prescription label is printed out at the home pharmacy and the prescription is filled by the home pharmacy from their drug inventory. The prescription label may contain various information such as the name, address, and phone number of the home pharmacy. The filled prescription is then ready to be picked up by the customer.

If the prescription is a hub-filled prescription, then the system generates a billing pre-claim and sends this information to the switch. The system then sends information about the prescription to the home pharmacy into a hub-fill holding queue. This information can include one or more of: Patient Name; Date of Birth; Phone Number; Rx#; Drug Name; NDC#; Drug Directions; Doctor Name; Prescription Expiration Date; Drug Schedule; Drug Manufacturer; Last Fill Date; Last Fill Quantity; and Quantity Remaining. Pharmacists or other staff members of the home pharmacy then process the prescriptions in the hub-fill holding queue. Such processing can include performing a review for drug interactions with other prescriptions filled for the specific patient.

After the hub-filled prescription is processed by the home pharmacy, billing information for the prescription (insurance claim) is sent to a Switch. The switch can distinguish between hub-filled prescriptions and home-filled prescriptions based on whether or not a pre-claim has already been sent to the switch by the hub pharmacy. For hub-filled prescriptions, the switch then submits billing information to the appropriate third party (PBM) using the NCPDP (National Council for Prescription Drug Programs) provider identification number of the hub pharmacy. The billing information can be sent to the switch by the home pharmacy directly or by the hub pharmacy. Next, the switch conveys information on whether the claim is allowed or denied back to the home pharmacy, either directly or through the hub pharmacy.

If the claim is allowed, the home pharmacy initiates the process of printing prescription labels. However, for hub-filled prescriptions, the label information is not printed out by the home pharmacy. Rather this information is provided to the hub pharmacy. This may be accomplished in a variety of ways. In an embodiment, a “dummy” printer is configured in the software of the home pharmacy, wherein printing to the “dummy” printer results in a data file being generated and forwarded to the hub pharmacy. The data file may include various pieces of information including one or more of: Patient Name; Patient Address; Patient Phone; Patient Date of Birth; Prescription Number; Drug Name; Directions for Use; Quantity of Medication Dispensed; NDC Number of Medication Dispensed; Refills Remaining on Medication; Days Supply of Medication; Doctor's Name; Doctor's DEA Number; Name of Third Party Payer; Patient Copay Amount; Amount Insurance Paid; Third Party Authorization Number; Pharmacist Initials; and If the Patient Wants Child Resistant Container or Not.

The hub pharmacy aggregates information about hub-filled prescriptions it receives over a predetermined amount of time, for example 1 day, and then puts all of these prescriptions into pharmacy specific batches for prescription filling purposes. Thus all the prescriptions that need to be delivered to a particular home pharmacy can be processed at one time by the hub pharmacy as a batch. The hub pharmacy processes the batch including filling the prescriptions and printing prescription labels. The prescription labels may include various information such as the name, address, and phone number of the home pharmacy. The prescription label can be configured to contain various types of information including custom instructions and the like.

In an embodiment, the hub pharmacy uses an automated system or robot to fill the prescription. By way of example, suitable automated filling systems include the SP 200 Robotic Prescription Dispensing System available from SCRIPTPRO®, Mission, Kans.; the Parata RDS available from Parata Systems, Durham, N.C.; the PharmASSIST system available from Innovation Associates, Johnson City, N.Y.; and the like.

The filled prescriptions are then delivered to the home pharmacies where the pharmacist can provide a final certification of the accuracy of the prescription. The filled prescription is then ready to be picked up by the customer.

After prescriptions are ready to be picked up by the customer, for either home-filled or hub-filled prescriptions, the system of the invention can optionally arrange to have the patient contacted to indicate that the prescription has been filled and is available for pick-up at the home pharmacy. This can be done in a variety of ways. For example, the system of the invention can telephone the patient and deliver an audio message or alternately can e-mail or fax the patient. Alternately, the system of the invention can prompt the home pharmacy to telephone the patient and deliver an audio message. The system can be set up to contact patients at any time of the day desired. The system can also be configured so that patients are not contacted by the system if they have requested this. In some embodiments, once the patient picks up the prescription, this information is relayed to the hub pharmacy so that records can be updated. When the patient comes to pick-up the prescription, the staff at the home pharmacy can provide any counseling and advice to the patient that is necessary.

In an embodiment, the patient is contacted the day the prescription has arrived at the home pharmacy. In an embodiment, the patient is contacted the day after the prescription has arrived at the home pharmacy. In an embodiment, the hub pharmacy scans records each day in a batch format to find out which prescriptions were delivered and then contacts the corresponding patients.

While not shown in the flowchart of FIG. 8, prescriptions that were previously enrolled for processing by the system of the invention but that are now out of refills are also placed in a home pharmacy out-of-refills processing queue. Staff at the home pharmacy can then call or fax the physician to authorize further refills. The home pharmacy may then fill the prescription out of the home pharmacy inventory regardless of whether or not the medication is on the hub formulary so that the prescription will be ready for the patient in time.

As described above, there are various tasks for the staff of the home pharmacy to perform and these can be managed by putting them in various queues such as the home-filled holding queue, the hub-fill holding queue, and the out-of-refills processing queue. Embodiments of the invention can help to ensure that these tasks are regularly and timely performed by the home pharmacy staff. By way of example, the hub pharmacy can send alerts to indicate that the records in the various queues need to be processed. Alerts can be sent via e-mail, pager, telephone, etc. The hub pharmacy can also cause message boxes to appear on the desktops of computers located at the home pharmacy to indicate that these tasks need to be performed. In an embodiment, the hub pharmacy system can require that these tasks be performed on a daily basis.

Some home pharmacies may not wish to use all of the features described herein. By way of example, some home pharmacies may only wish to take advantage of the timing and billing features and desire to fill all prescriptions themselves. Embodiments of the invention can be configured to provide only certain functions to the home pharmacies.

Billing Procedures

As described above, billing information is sent by a switch to a third party using either the NCPDP number of the home pharmacy (for home-fill prescriptions) or the NCPDP number of the hub pharmacy (for hub-fill prescriptions). Where the NCPDP number of the home pharmacy has been provided, the third party (such as an insurance company) sends the payment for the prescription to the home pharmacy. Where the NCPDP number of the hub pharmacy has been provided, the third party (such as an insurance company) sends the payment to the hub pharmacy. The home pharmacy collects the co-pay amount from the patient when the customer picks up the prescription.

Payment schemes between the hub pharmacy and home pharmacy can be set up in a variety of ways. In an embodiment, the hub pharmacy agrees to pay the home pharmacy a consultation fee per hub-filled prescription based on a sliding scale that takes into account factors such as the volume of prescriptions filled for the particular home pharmacy by the hub pharmacy. Periodically, such as every two weeks, a billing report is sent to the home pharmacy. The hub pharmacy calculates the total amount of consultation fees for the period minus the amount of co-pays collected by the home pharmacy. If the resulting amount is positive, then the hub pharmacy provides payment to the home pharmacy in that amount. If the resulting amount is negative, then a bill is sent to the home pharmacy for this amount.

Various procedures can optionally be followed to ensure billing accuracy. By way of example, when the hub pharmacy is getting paid from the third party, the hub pharmacy provides billing authorization data to the home pharmacy that indicates the hub pharmacy will receive third party payment. This billing authorization data shows up on the home pharmacy's computer system. This billing authorization data also prints out on the patient's pharmacy receipt. If the home pharmacy sees the billing authorization data that indicates the hub pharmacy will receive third party payment on their pharmacy computer system, or on a patient's pharmacy receipt, the home pharmacy will know that the hub pharmacy got paid for the prescription.

As another example of an optional procedure, the prescription label information will not be printed out by the hub pharmacy unless the billing authorization data referred to above indicates that the hub pharmacy will receive third party payment. If the label information is not printed out, the hub pharmacy will not fill the prescription.

As a further example of an optional procedure, every day, a log of prescriptions that were paid to the hub pharmacy is generated by the hub pharmacy and is compared to a log of prescriptions that have been filled by the hub pharmacy. If any discrepancies are found, the claim can be re-billed to the correct pharmacy. This ensures that only prescriptions that the hub pharmacy receives payment for are filled by the hub pharmacy and only prescriptions that the home pharmacy receives payment for are filled by the home pharmacy.

Return Procedures

Sometimes, patients fail to pick-up their prescriptions from the home pharmacy in a timely manner. Typically, these prescriptions are held on to for a time and then returned to inventory. Embodiments of the invention include optional procedures for handling returns.

By way of illustration, prescriptions that are filled by the hub pharmacy will have a return date (“return to stock date”) printed on the receipt label. If a patient does not pick up the prescription by this date, the home pharmacy must reverse the prescription claim by sending information to the switch and then send the medication back to the hub pharmacy. The return to stock date can be calculated in various ways. For example, if a prescription is for a days supply of 0-33 days, it can have a return to stock date of 22 days past the date the prescription was filled. Table 1 below outlines exemplary return to stock (RTS) dates for different ranges of days supplies.

TABLE 1

| Days Supply | RTS Date |
|---|---|
| 0-33 | 22 days |
| 34-59 | 24 days |
| 60-63 | 29 days |
| 64-89 | 34 days |
| 90-99 | 37 days |
| >99 | 42 days |

As an example, when the returned product arrives at the hub pharmacy, the prescriptions are scanned into a log identifying the date that the product arrived at the hub pharmacy, the prescription number, and the date that the prescription was originally filled. At the end of every month, two reports are generated by the hub pharmacy. The reports compare the prescription reversal information from the billing statement to the log of prescriptions that were returned to the hub pharmacy. The first report shows prescriptions that were reversed from the insurance, but not returned to the hub pharmacy. The second report shows prescriptions that were returned to the hub pharmacy, but were not reversed from the insurance.

Corrective action can then be taken by the staff of the home pharmacy, the staff of the hub pharmacy, or automatically. For example, the hub pharmacy can charge the home pharmacy for the cost of the drug for prescriptions that were reversed from the insurance, but were not returned to the hub pharmacy. Also, a list of prescriptions that were returned to the hub pharmacy, but were not reversed from the insurance, can be provided to the home pharmacy, so that they can reverse the claim from the insurance.

Nursing Home Procedures

Embodiments of the invention can be used in association with different types of health provider environments to serve both in-patients and out-patients. By way of example, embodiments of the invention can be used to fill prescriptions not only for people who attend out-patient clinics, but also for people who are in care facilities such as nursing homes, assisted-living environments, hospice care, hospitals, retirement community health centers, and the like.

Facilities such as nursing homes may have special requirements with respect to filling the prescriptions of patients in their care. For example, in the interest of efficiency, the nursing home may request that all prescriptions for patients in their care be filled at the same time and be available for pick-up from the home-pharmacy as a batch. The day that prescriptions are picked-up or delivered for all their in-patients can be referred to as the “change over” day. Systems and methods of the invention can be configured to accommodate these types of special needs.

As an example, procedures that could be used in the case of a nursing home will be discussed. New prescriptions for nursing home patients are filled the first time at the home pharmacy with enough days supply to last the patient until the “change over” day. The staff of the home pharmacy can then enroll the prescription(s) for further processing by the system. For example, the staff can enter the prescription number into the software and then mark it as being active.

At a predetermined time, such as about a week before the nursing home's change over day, prescriptions for that nursing home are ordered by the hub pharmacy and put into a batch. After a period of time, the batch is closed and pre-claims for the prescriptions are sent to the switch. The prescription information for the batch is then sent into a nursing-home holding queue on the home pharmacy's computer. These prescriptions can be identified in various ways. For example, these prescriptions can be identified by having “NH” in a comment field for the prescription.

Staff at the home pharmacy then process all prescriptions that were placed in the nursing-home holding queue by the end of the day that they appear in the queue. After staff at the home pharmacy process the prescriptions, claim information is sent to the switch directly or through the hub pharmacy. The claim information is matched up with the pre-claims and the prescription is then billed using the hub pharmacy's NCPDP number. When the claim is billed using the hub pharmacy's NCPDP number, the hub pharmacy sends an authorization number back to the home pharmacy. The label information is then collected in a batch for dispensing at the hub pharmacy.

The prescriptions are then filled and the prescription labels are printed out at the hub pharmacy. The prescription is packaged in a type of unit dose packaging. The type of packaging can vary depending on what packaging the nursing home requests. When all of the prescriptions are filled for the nursing home, they are sent to the home pharmacy via courier. The home pharmacy then delivers the prescriptions to the nursing home on their change over day or has the nursing home pick-up the prescriptions. In this manner, all the of the prescriptions for all of the patients in the nursing home can be provided at one time as opposed to individually. Billing and returns can be handled the same as described above.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “adapted and configured” describes a system, apparatus, or other structure that is constructed or configured to perform a particular task or adopt a particular configuration to. The phrase “adapted and configured” can be used interchangeably with other similar phrases such as arranged and configured, constructed and arranged, adapted, constructed, manufactured and arranged, and the like.

The invention has been described with reference to various specific and preferred embodiments and techniques. However, it should be understood that many variations and modifications may be made while remaining within the spirit and scope of the invention.

1. A method for filling refill prescriptions for one or more home pharmacies by a hub pharmacy comprising:
enrolling the refill prescription records of one or more home pharmacies;
calculating a fill time for refill prescriptions; and
filling the refill prescriptions;
wherein enrolling and calculating are performed by the hub pharmacy.

2. (canceled)

3. The method of claim 1, wherein the step of enrolling includes one or more of:
determining if the prescription record has refills;
determining if the prescription record has been flagged for non-enrollment;
determining if the prescription record is for a prescription lasting≧predetermined period of time; and
determining whether the prescription record is for a medication that is on a formulary of the hub pharmacy.

4. The method of claim 3, wherein the step of enrolling comprises:
determining if the prescription record has refills;
determining if the prescription record has been flagged for non-enrollment;
determining if the prescription record is for a prescription lasting≧predetermined period of time; and
determining whether the prescription record is for a medication that is on a formulary of the hub pharmacy.

5. The method of claim 3, the predetermined length of time comprising one month.

6. The method of claim 1, wherein the step of filling is performed by the hub pharmacy if the prescription is for a medication on the hub pharmacy formulary, wherein the step of filling is performed by the home pharmacy if the prescription is for a medication that is not on the hub pharmacy formulary.

7. The method of claim 1, the step of filling comprising sending information about the refill prescription into a holding queue for further processing by the home pharmacy.

8. The method of claim 7, further processing comprising checking for drug interactions.

9. The method of claim 1, wherein the step of calculating comprises:
setting the fill time to a date when a previous fill is approximately 100% consumed if the previous fill was provided by the method of the invention.

10. The method of claim 1, wherein the step of calculating comprises:
setting the fill time to a date when a previous fill is approximately 70% to 80% consumed if the previous fill was not provided by the method of the invention.

11. The method of claim 1, wherein the step of calculating comprises:
setting the fill time to a date when a previous fill is approximately 75% consumed if the previous fill was not provided by the method of the invention.

12. The method of claim 1, further comprising the step of notifying a patient that the prescription has been filled.

13. The method of claim 12, wherein the patient is notified via telephone.

14. The method of claim 6, further comprising the step of billing a third party for the filled prescription.

15. The method of claim 14, billing the third party comprising submitting claim information to a switch.

16. The method of claim 15, wherein submitting claim information to the switch is performed by the home pharmacy.

17. The method of claim 15, billing the third party comprising submitting a pre-claim to the switch from the hub pharmacy if filling is performed by the hub pharmacy.

18. The method of claim 17, the switch determining whether filling was performed by the hub pharmacy or the home pharmacy by searching for a pre-claim.

19. A system for processing refill prescriptions comprising:
a hub pharmacy comprising a hub formulary and a processing unit; and
one or more home pharmacies each comprising a prescription information database comprising records of prescriptions, the home pharmacies in electronic communication with the hub pharmacy;
the processing unit adapted and configured to query the prescription information databases to find records of prescriptions to be refilled;
the hub pharmacy adapted and configured to fill prescriptions for medications that are on the hub formulary;
the one or more member pharmacies adapted and configured to fill prescriptions for medications that are not on the hub formulary;
the hub pharmacy adapted and configured to submit billing information to third parties for prescriptions that are filled by the hub pharmacy.

20. The system of claim 19, the hub pharmacy comprising an automated prescription dispenser.

21. The system of claim 19, the processing unit adapted and configured to determine the appropriate time to fill prescriptions for the records of prescriptions to be refilled.

22. The system of claim 19, the processing unit adapted and configured to find records of prescriptions that have not been flagged for non-enrollment and that are for a prescription lasting≧a predetermined period of time.

23. The system of claim 22, the predetermined length of time comprising one month.
